(19) United States
(12) Patent Application Publication
(10) Pub. No.: US 2025/0160631 A1
(43) Pub. Date: May 22, 2025

(54) DEVICES, SYSTEMS, AND METHODS FOR HANGING FLUID RESERVOIRS IN SYSTEMS TO SUPPLY FLUIDS TO AN ENDOSCOPE

(71) Applicant: Boston Scientific Scimed, Inc., Maple Grove, MN (US)

(72) Inventors: Austin Grant Johnson, Hudson, MA (US); Ryan V. Wales, Northborough, MA (US); Rosangel Aimee Ramos Espinoza, Allston, MA (US); Scott Edward Corbeil, Litchfield, NH (US)

(73) Assignee: Boston Scientific Scimed, Inc., Maple Grove, MN (US)

(21) Appl. No.: 18/947,444

(22) Filed: Nov. 14, 2024

Related U.S. Application Data
(60) Provisional application No. 63/599,674, filed on Nov. 16, 2023.

(51) Int. Cl. A61B 1/015; A61B 50/13; A61J 1/10; A61J 1/16

(52) U.S. Cl. CPC A61B 1/015; A61B 50/13; A61J 1/16; A61J 1/10

(57) ABSTRACT

Coupling members and systems for hanging a fluid reservoir relative to an endoscope cart. An illustrative coupling member may comprise an elongate body extending from a first end to a second end. The elongate body may be configured to extend through an opening of a flexible reservoir and around a bar of an endoscope cart.

### CROSS-REFERENCE TO RELATED APPLICATIONS

This application claims the benefit of U.S. Provisional Patent Application Ser. No. 63/599,674 filed on Nov. 16, 2023, the disclosure of which is incorporated herein by reference.

### FIELD

This disclosure relates generally to medical fluid containers and methods, and particularly to container and tube sets to supply fluid and/or gas to an endoscope

### BACKGROUND

Conventionally, endoscope devices have been widely used for performing diagnostic and/or therapeutic treatments. During endoscopic procedures, physicians may use a combination of air, irrigation, and lens wash as a means of flushing debris, cleaning optics, and insufflating the working lumen. For example, sterile water may be used to irrigate the working lumen and/or clear organic matter (e.g., clots, fecal matter, or other masses) from the area of interest during the procedure. This may be achieved by spraying a low velocity jet of water from an orifice at the tip of the endoscope. Further, during endoscopic procedures, the video lens at the distal end of the endoscope, which is used to navigate and visualize target tissues, may be prone to becoming fouled with blood, mucous, and other debris during the procedure. Physicians need to be able to clear the lens and working lumen throughout the procedure. To generate lens wash, a connector is coupled to an endoscope umbilical via a tube set. The tube set may translate air from the endoscope umbilical to the water container or pressure vessel. A water pickup tube within the container or pressure vessel is fluid contact with the water and coupled to the connector. This allows for the pressure building in the water bottle or pressure vessel to translate the water up the water tube to the distal tip of the endoscope to clean the endoscope lens.

Endoscope fluid delivery for both lens wash and irrigation processes may include a flexible tube set with a threaded cap which connects to a one-liter (L) semi-rigid bottle of sterile water for irrigation. Commercially available tube sets may be available as either traditional two bottle tube sets or hybrid tubing sets. Traditional tubing sets consist of two separate tube sets which connect to two individual bottles of sterile water. One tube set connects to a one-liter bottle of sterile water while a second tube set is connected to second bottle for irrigation. Hybrid tubing sets, in comparison, include of a single tube set which connects to a single one-liter bottle water source, and this single system enables both irrigation and lens wash functions.

In some cases, it may be desirable to leverage off-the-shelf solution bags of sterile water or normal saline instead of bottles. There may be many advantages to using solution bags over current bottle driven systems, including, but not limited to, enabling the user to tailor the volume of the fluid reservoir to their practice needs. For example, a user can use a one-, two-, or three-liter solution bag, which are readily available to them, thus reducing or eliminating the need to change one-liter bottles over and over throughout a procedural day.

Solution bags may need to be hung. However, many endoscopy suites have space limitations, and as such have no space for a separate IV pole to hang the tubing set solution bag. As such, there is a need for a hanger that will enable the solution bag to be hung from an endoscopy cart, which typically houses capital equipment and the one-liter bottles used in current commercially available tubing sets. It is with these considerations in mind that the improvements of the present disclosure may be useful.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary. Accordingly, while the disclosure is presented in terms of aspects or embodiments, it should be appreciated that individual aspects can be claimed separately or in combination with aspects and features of that embodiment or any other embodiment.

In a first example, a coupling member for hanging a fluid reservoir relative to an endoscope cart may comprise an elongate body extending from a first end to a second end. The elongate body may be configured to extend though an opening of a flexible reservoir and around a bar of an endoscope cart.

Alternatively or additionally to any of the examples above, in another example, the elongate body may comprise a flexible wire.

Alternatively or additionally to any of the examples above, in another example, the coupling member may further comprise a coating disposed over the elongate body.

Alternatively or additionally to any of the examples above, in another example, free end regions of the elongate body may be configured to be twisted together to form a looped region.

Alternatively or additionally to any of the examples above, in another example, the elongate body may further comprise an insertion head adjacent to the second end thereof and plurality of teeth formed in a surface of the elongate body between the first end and the second end.

Alternatively or additionally to any of the examples above, in another example, the first end of the elongate body may be configured to be inserted into the insertion head to form a looped region.

Alternatively or additionally to any of the examples above, in another example, a pawl within the insertion head may be configured to engage at least one tooth of the plurality of teeth.

Alternatively or additionally to any of the examples above, in another example, the elongate body may comprise a flexible material.

Alternatively or additionally to any of the examples above, in another example, the flexible material may be configured to be knotted to form a looped region.

Alternatively or additionally to any of the examples above, in another example, the elongate body may be a bistable spring.

Alternatively or additionally to any of the examples above, in another example, the elongate body may be movable from a first elongated configuration to a second curled configuration defining a looped region.

Alternatively or additionally to any of the examples above, in another example, in the first elongated configuration, the elongate body may be curved between a first lateral side and a second lateral side.

Alternatively or additionally to any of the examples above, in another example, in the second curled configuration, the first end of the elongate body may overlap the second end of the elongate body.

Alternatively or additionally to any of the examples above, in another example, the elongate body may comprise a metallic core and a polymeric coating.

Alternatively or additionally to any of the examples above, in another example, the coupling member may further comprise a hook coupled to the elongate body.

In another example a coupling member for hanging a fluid reservoir relative to an endoscope cart may comprise an elongate body extending from a first end to a second end, a first buckle secured adjacent to the first end of the elongate body, a second buckle secured adjacent to the second end of the elongate body, and a hook coupled to the elongate body. The elongate body may be configured to wrap around a region of an endoscope cart.

Alternatively or additionally to any of the examples above, in another example, the elongate body may comprise a flexible strap.

Alternatively or additionally to any of the examples above, in another example, the first buckle may be releasably coupled to the second buckle.

Alternatively or additionally to any of the examples above, in another example, a length of the elongate body is adjustable.

In another example, a coupling member for hanging a fluid reservoir relative to an endoscope cart may comprise a generally planar body portion extending from a first end to a second end, a hook region extending from the second end of the generally planar body portion, the hook region including a curved region, a securement member extending from a back surface of the generally planar body portion, the securement member including an opening extending therethrough, and a bracket extending from the back surface of the generally planar body portion. The hook region may be configured to receive a portion of a fluid reservoir.

Alternatively or additionally to any of the examples above, in another example, the coupling member may further comprise at least one arm extending from a lateral side of the generally planar body portion.

Alternatively or additionally to any of the examples above, in another example, the at least one arm may be configured to receive a tubing.

Alternatively or additionally to any of the examples above, in another example, the bracket may be generally “L” shaped.

Alternatively or additionally to any of the examples above, in another example, the bracket may include a first leg extending generally orthogonal to a plane of the generally planar body portion and a second leg extending generally parallel to the plane of the generally planar body portion.

Alternatively or additionally to any of the examples above, in another example, the second leg may be spaced a distance from the back surface of the generally planar body portion.

Alternatively or additionally to any of the examples above, in another example, the bracket may be configured to releasably secure to a shelf of an endoscope cart.

Alternatively or additionally to any of the examples above, in another example, the coupling member may be a single monolithic structure.

In another example, a coupling member for hanging a fluid reservoir relative to an endoscope cart may comprise an elongate body extending from a first end to a second end, the elongate body movable from a first elongated configuration to a second looped configuration. The elongate body may be configured to extend though an opening of a flexible reservoir and around a bar of an endoscope cart.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various exemplary embodiments and together with the description serve to explain the principles of the present disclosure.

FIG. 1 depicts components of an endoscope;

FIG. 2 depicts components of an endoscope system with endoscope, light source, light source connector, water reservoir, and tubing assembly for air and lens wash fluid delivery;

FIG. 3 depicts another illustrative endoscope system having an alternative fluid supply system;

FIG. 4 is a schematic perspective view of an illustrative cart that may be used to hold or mount capital equipment during an endoscopic procedure;

FIG. 5 is a perspective view of a portion of the cart of FIG. 4 including an upper shelf of the endoscope cart having a push bar with a reservoir may be releasably secured thereto;

FIG. 6 is a perspective view of an illustrative coupling member;

FIG. 7 is a perspective view of the illustrative coupling member of FIG. 6 with an optional hook;

FIG. 8A is a perspective view of another illustrative coupling member in a first elongated configuration;

FIG. 8B is a perspective view of the illustrative coupling member of FIG. 8A in a second looped configuration;

FIG. 9 is a perspective view of another illustrative coupling member in a first elongated configuration;

FIG. 10A is a perspective view of another illustrative coupling member in a first elongated configuration;

FIG. 10B is an end view of the illustrative coupling member of FIG. 10A in a second looped configuration;

FIG. 11 is a perspective view of another illustrative coupling member;

FIG. 12 is a partial front view of the illustrative cart having the coupling member of FIG. 11 secured thereto;

FIG. 13 is a front perspective view of another illustrative coupling member;

FIG. 14 is a rear perceptive view of the illustrating coupling member of FIG. 14;

FIG. 15 is a cross-sectional view of the illustrative coupling member of FIGS. 13-14 taken at line 15-15 of FIG. 14; and

FIG. 16 is a schematic view of the illustrative coupling member of FIGS. 13-15 secured to a cart and holding a reservoir.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an exemplary medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, the same or similar reference numbers will be used through the drawings to refer to the same or like parts.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” Further, as used herein, the terms “about,” “approximately” and “substantially” indicate a range of values within +/−10% of a stated or implied value. Additionally, terms that indicate the geometric shape of a component/surface refer to exact and approximate shapes.

Embodiments of the present disclosure are described with specific reference to a bottle (e.g., container, reservoir, or the like) and tube assembly or set. It should be appreciated that such embodiments may be used to supply fluid and/or gas to an endoscope, for a variety of different purposes, including, for example to facilitate insufflation of a patient, lens washing, and/or to irrigate a working channel to aid in flushing/suctioning debris during an endoscopic procedure.

Although the present disclosure includes descriptions of a container and tube set suitable for use with an endoscope system to supply fluid and/or gas to an endoscope, the devices, systems, and methods herein could be implemented in other medical systems requiring fluid and/or gas delivery, and for various other purposes.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Conventionally, endoscope devices have been widely used for performing diagnostic and/or therapeutic treatments. During endoscopic procedures, physicians may use a combination of air, irrigation, and lens wash as a means of flushing debris, cleaning optics, and insufflating the working lumen. For example, sterile water may be used to irrigate the working lumen during the procedure. Further, during endoscopic procedures, the video lens at the distal end of the endoscope, which is used to navigate and visualize target tissues, may be prone to becoming fouled with blood, mucous, and other debris during the procedure. To generate lens wash, a connector is coupled to an endoscope umbilical via a tube set. The tube set may translate air from the endoscope umbilical to the water container or pressure vessel. A water pickup tube within the container or pressure vessel is fluid contact with the water and coupled to the connector. This allows for the pressure building in the water bottle or pressure vessel to translate the water up the water tube to the distal tip of the endoscope to clean the endoscope lens. The tube set used for providing irrigation fluid and/or lens wash fluid may be commonly used for a period of 24 hours across multiple endoscopic procedures. However, the same endoscope is not used for multiple patients and must be switched out between procedures. When the procedure is over, the connector is disconnected from the umbilical. The water remaining within the tube set may spill on the floor upon disconnection. Additionally, the residual pressure within the connector or pressure vessel may cause a siphon vacuum to be pulled, thus spilling all of the water in the container/pressure vessel out onto the floor via the tube set. Having a means of preventing water leaking and/or siphoning can prevent the user from needing to clean water on the floor, prevent damage to capital equipment adjacent or under the tubing connector, or requiring the user to get an additional water container or refill the water container for the next procedure. Disclosed herein are devices and systems to prevent siphoning and/or leaks of water from the water bottle and/or tube set after the procedure

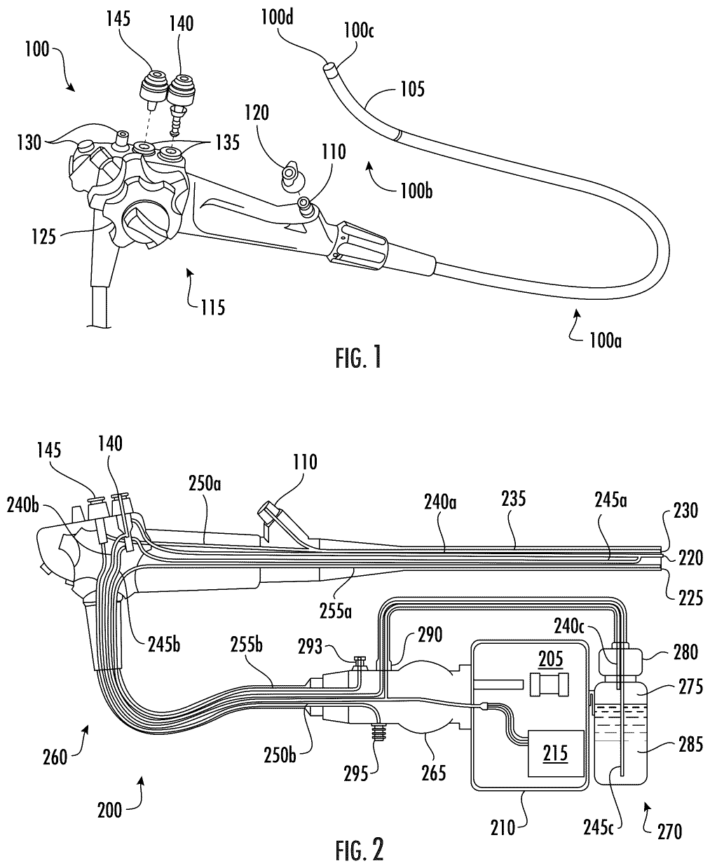

With reference to FIGS. 1-2, an exemplary endoscope 100 and system 200 are depicted that may comprise an elongated shaft 100a that is inserted into a patient. A light source 205 feeds illumination light to a distal portion 100b of the endoscope 100, which may house an imager (e.g., CCD or CMOS imager) (not shown). The light source 205 (e.g., lamp) is housed in a video processing unit 210 that processes signals that are input from the imager and outputs processed video signals to a video monitor (not shown) for viewing. The video processing unit 210 also serves as a component of an air/water feed circuit by housing a pressurizing pump 215, such as an air feed pump, in the unit.

The endoscope shaft 100a may include a distal tip 100c provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 extends along the shaft 100a to a proximal channel opening 110 positioned distal to an operating handle 115 of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115. In addition, the handle 115 is provided with dual valve wells 135. One of the valve wells 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2). The other valve well 135 receives a suction valve 145 for operating a suction operation. A suction supply line 250a runs distally from the suction valve 145 along the shaft 100a to a junction point in fluid communication with the working channel 235 of the endoscope 100.

The operating handle 115 is electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) is fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The detachable gas/lens wash connection 290 may be detachable from the connector portion 265 and/or the gas supply tubing 240c. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, passes through the top 280 of the reservoir 270 to the same detachable connection 290 as the gas supply tubing 240c on the connector portion 265. In other embodiments, the connections may be separate and/or separated from each other. The connector portion 265 also has a detachable irrigation connection 293 for irrigation supply tubing (not shown) running from a source of irrigation water (not shown) to the irrigation feed line 255b in the umbilical 260. The detachable irrigation connection 293 may be detachable from the connector portion 265 and/or the irrigation supply tubing (not shown). In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100. The detachable suction connection 295 may be detachable from the connector portion 265 and/or the suction feed line 250b and/or the vacuum source.

The gas feed line 240b and lens wash feed line 245b are fluidly connected to the valve well 135 for the gas/water valve 140 and configured such that operation of the gas/water valve 140 in the well controls supply of gas or lens wash to the distal tip 100c of the endoscope 100. The suction feed line 250b is fluidly connected to the valve well 135 for the suction valve 145 and configured such that operation of the suction valve in the well controls suction applied to the working channel 235 of the endoscope 100.

Referring to FIG. 2, an exemplary operation of an endoscopic system 200, including an endoscope such as endoscope 100 above, is explained. Air from the air pump 215 in the video processing unit 210 is flowed through the connector portion 265 and branched to the gas/water valve 140 on the operating handle 115 through the gas feed line 240b in the umbilical 260, as well as through the gas supply tubing 240c to the water reservoir 270 via the connection 290 on the connector portion 265. When the gas/water valve 140 is in a neutral position, without the user's finger on the valve, air is allowed to flow out of the valve to atmosphere. In a first position, the user's finger is used to block the vent to atmosphere. Gas is allowed to flow from the valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve, allowing pressure of the air passing from the air pump 215 to rise in the water reservoir 270. Pressurizing the water source forces water out of the lens wash tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash tubing 245c, the air pump 215 replaces lost air supply in the reservoir 270 to maintain a substantially constant pressure, which in turn provides for a substantially constant lens wash flow rate. In some embodiments, a filter (not shown) may be placed in the path of the gas supply tubing 240c to filter-out undesired contaminants or particulates from passing into the water reservoir 270. In some embodiments, outflow check valves or other one-way valve configurations (not shown) may be placed in the path of the lens wash supply tubing to help prevent water from back-flowing into the reservoir 270 after the water has passed the valve.

A relatively higher flow rate of irrigation water is typically required compared to lens wash, since a primary use is to clear the treatment area in the patient of debris that obstructs the user's field of view. Irrigation is typically achieved with the use of a pump (e.g., peristaltic pump), as described. In embodiments with an independent water source for irrigation, tubing placed in the bottom of a water source is passed through the top of the water source and threaded through the head on the upstream side of the pump. Tubing on the downstream side of the pump is connected to the irrigation feed line 255b in the umbilical 260 and the irrigation supply line 255a endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical, and down the irrigation supply line in the shaft 100a of the endoscope to the distal tip 100c. In order to equalize the pressure in the water source as water is pumped out of the irrigation supply tubing, an air vent (not shown) may be included in the top of the water reservoir. The vent allows atmospheric air into the water source preventing negative pressure build-up in the water source, which could create a vacuum that suctions undesired matter from the patient back through the endoscope toward the water source. In some embodiments, outflow check valves or other one-way valve configurations (not shown), similar to the lens wash tubing 245c, may be placed in the path of the irrigation supply tubing to help prevent back-flow into the reservoir after water has passed the valve.

It is contemplated that other arrangements for the fluid sources may be used as desired. For example, in some cases, water for irrigation and lens wash may come from a same container. Some illustrative systems and method to supply fluids to the endoscope are described in commonly assigned U.S. Patent Application No. 63/419,900, titled DEVICES, SYSTEMS, AND METHODS TO SUPPLY FLUIDS TO AN ENDOSCOPE, the disclosure of which is hereby incorporated by reference.

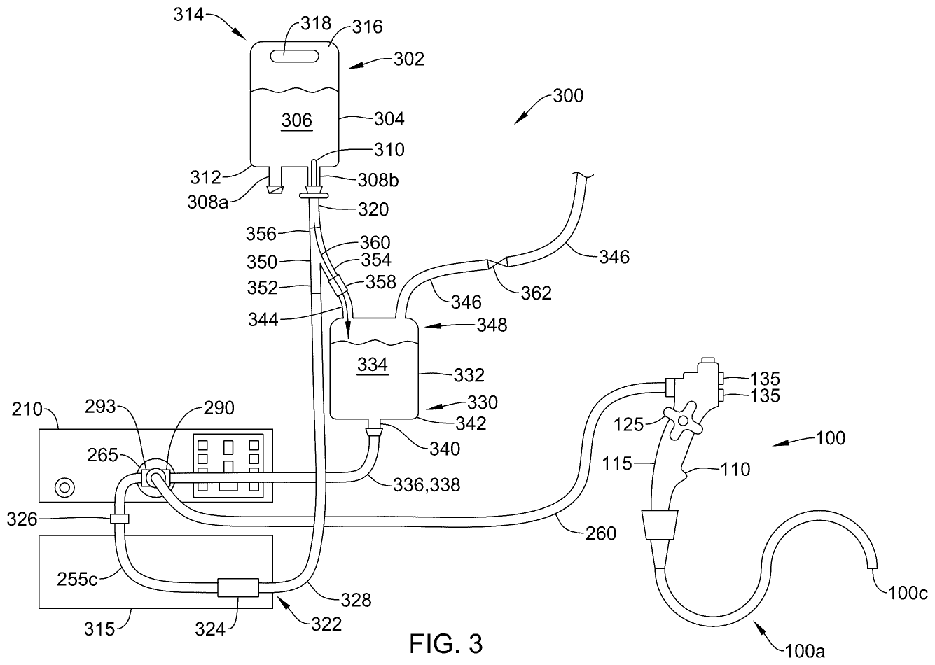

FIG. 3 depicts a schematic view of another illustrative endoscopic system 300 which may reduce the number of water reservoir changes and/or reduce opportunities for contamination during replacement of the water reservoir(s). The system 300 may include a number of advantages over the current bottle system described above. The system 300 may include components similar to the endoscope and endoscope systems described with regard to FIGS. 1-2; however, not all features may be described or shown here.

Generally, the system 300 may include a first reservoir 302 and a second reservoir 330. The first reservoir 302 may be configured to supply water or fluid for both irrigation (e.g., via the first reservoir 302) and lens wash (e.g., via the second reservoir 330). This may allow a single fluid source to be used to provide fluid for both irrigation and lens wash. While not explicitly shown, the reservoirs 302, 330 may include printed lines, numbers, or other visual indicia to allow a user to easily determine how much fluid is left in the reservoirs 302, 330.

The first reservoir 302 may include a first container 304 configured to hold a first volume of fluid 306. In the illustrated embodiment, the first container 304 is fluidly coupled to the upstream irrigation supply tubing 328 and is configured to provide fluid for irrigation to the endoscope 100. Generally, the irrigation supply tubing 328 may be a water or fluid supply line or tube for supplying water or other fluid to an endoscope. Additionally, the first container 304 may be selectively fluidly coupled to a second fluid reservoir 330. The second reservoir 330 may include a second container 332 configured to hold a second volume of fluid 334. In the illustrated embodiment, the second container 332 is fluidly coupled to the gas and lens wash supply tubing 336, 338 and is configured to provide fluid for lens wash to the endoscope 100. Generally, the lens wash supply tubing 338 may be a water or fluid supply line or tube for supplying water or other fluid to an endoscope. The gas and lens wash supply tubing 336, 338 may be coaxially arranged. For example, the gas supply tubing 336 may define a lumen that is sufficiently large in diameter to encompass a smaller diameter lens wash tubing 338, coaxially received within the gas supply tubing 336, as well as provide air to the water source in an annular space surrounding the lens wash tubing 338 to pressurize the second reservoir 330. The lens wash supply tubing 338 may be configured to exit the lumen defined by the coaxial gas supply tubing 336 in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion 265. In other embodiments, the gas and lens wash supply tubing 336, 338 may be arranged in a side-by-side arrangement.

The first and second containers 304, 332 may be formed from one or more layers of a lightweight, flexible material, such as, but not limited to, low density polyethylene (LDPE), thermoplastic polyurethane (TPU), silicone, polyethylene terephthalate (PET), aluminum, nylon, polyethylene (PE), plasticized polyvinyl chloride (PVC), or combinations thereof, etc. In some embodiments, the first and second containers 304, 332 may be entirely translucent, entirely opaque, or combinations thereof. In some cases, the first and second containers 304, 332 may be a flexible bag analogous to those utilized to deliver intravenous replacement fluid in clinical settings (for example, an intravenous (IV) fluid bag). Such bags may be readily available and familiar to the clinician as they are widely used in various sizes. The volume of the first and second containers 304, 332 may be variable. For example, the volume of the first container 304 and/or the second container 332 may be 500 milliliters (mL) or greater, 1000 mL or greater, 2000 mL or greater, 3000 mL, 4000 mL or greater, etc. The volume may be less than 500 mL or greater than 4000 mL, as desired. One or both of the first and second reservoirs 302, 330 may be pre-filled (e.g., prior to entering the procedure suite or at the time of manufacturing) with water or other fluid. In some cases, the clinician may select the reservoir(s) 302, 330 from a plurality of differently sized available reservoirs based on the number and/or types of procedures expected for a typical day or the specific day. In the illustrated embodiments, the first reservoir 302 may supply fluid to the second reservoir 330. By selecting a first reservoir 302 having a volume large enough to accommodate an entire day of procedures, the need for replacing the sterile fluid source (e.g., the first reservoir 302) may be reduced or eliminated. In some cases, the first reservoir 302 may be used to periodically refill the second reservoir 330. Thus, the volume of the first reservoir 302 may be greater than the volume of the second reservoir 330, although this is not required. It is further contemplated that, in some embodiments, one or both of the first or second reservoirs 302, 330 may be a rigid bottle.

It is contemplated that flexible bags may utilize less plastic (or other material) than a bottle designed to hold a similar amount of fluid. Thus, the use of a flexible bag as a fluid reservoir 302, 330 may increase the level of environmental sustainability of the system 300. For example, if the user sets up the system with a 3000 mL (3 liter) bag reservoir 302 and therefore does not need to utilize three individual one-liter bottles, a significant reduction of waste may be realized. It is further contemplated that when disposed of or discarded, a flexible bag reservoir may occupy less volume than a bottle capable of holding an equivalent amount of fluid.

The first reservoir 302 may further include one or more ports 308a, 308b, such as, but not limited to a spike port or a septum port, extending from and in selective fluid communication with an interior of the first container 304. The ports 308a, 308b may be formed as a monolithic structure with the first container 304. The ports 308a, 308b may be generally tubular structures with each port 308a, 308b defining a lumen extending therethrough. The lumens of the ports 308a, 308b may be configured to selectively fluidly couple the interior of the first container 304 with another component, such as, but not limited to, a fluid or water supply tube. In some embodiments, the ports 308a, 308b may be positioned adjacent to a bottom end 312 of the first reservoir 302. However, this is not required. The ports 308a, 308b may be positioned in other locations, as desired. If the ports 308a, 308b are positioned at a location other than the bottom end 312 of the first container 304, a dip tube or tube extension may be required to access the fluid at the bottom of the first container 304. In some cases, at least one port 308b may be configured to be coupled to the upstream irrigation tubing (or water supply tube) 328 while another port 308a may be configured to allow the user to add additives to the fluid 306. In other examples, the upstream irrigation tubing 328 may be coupled to the first port 308a while the second reservoir 330 is in fluid communication with the second port 308b. While the first reservoir 302 is illustrated as including two ports 308a, 308b, the first reservoir 302 may include one port or more than two ports, as desired.

While not explicitly shown, the ports 308a, 308b may each include a removable cap or seal configured to form a fluid tight seal with the port 308a, 308b. The removable cap or seal may help to maintain the sterility of the ports 308a, 308b. The removable cap or seal may be coupled to a free end of the ports 308a, 308b using a number of different techniques. For example, the cap or seal may be coupled to the port 308a, 308b using a threaded engagement, a friction fit, a snap fit, etc. In other instances, the cap or seal may be removed through a twisting motion configured to break the cap or seal from the port 308a, 308b. Once the cap or seal has been removed, the port 308a, 308b may be pierced with a spike tip or spike port adaptor 310 that is coupled to the upstream irrigation tubing 328. For example, in addition to the removable cap or seal, the port 308a, 308b may include an internal seal disposed within a lumen of the port 308a, 308b that may be punctured or pierced by the spike port adaptor 310. The internal seal may be configured to prevent fluid 306 from leaking from the first container 304 prior to the spike port adaptor 310 being inserted into the port 308a, 308b. In some embodiments, the internal seal may be self-sealing such that upon removal of the spike port adaptor 310 fluid is prevented from leaking from the port 308a, 308b. The outer surface of the spike port adaptor 310 may form an interference fit with the inner surface of the port 308a, 308b. The fit and/or coupling between the spike port adaptor 310 and the port 308a, 308b may be sufficient to remain in place when the irrigation supply tube 328, branched connector 350, and/or other tubing sets are coupled to the spike port adaptor 310. It is contemplated that the spike port adaptor 310 may be inserted into one of the ports 308a, 308b utilizing universally used aseptic techniques such as those used with IV fluid bags. This may help reduce infection risk by maintaining sterile components, not introducing contaminants into the fluid 306, etc. It is further contemplated that additives may be added to the fluid 306 using similar aseptic techniques via one of the ports 308a, 308b.

The first reservoir 302 may include a handle 316 positioned adjacent to a top portion 314 thereof. The handle 316 may define an opening or through hole 318 for receiving a hand or hook therethrough to carry the first reservoir 302. In some cases, the handle 316 may include an undulating surface configured to provide a more ergonomic grip for the user. It is contemplated that the handle 316 may be formed from a similar material as the first container 304 or a different material, as desired. In some examples, the handle 316 may be formed from polyethylene terephthalate (PET), polypropylene (PP), etc. The handle 316 may allow the first reservoir 302 to be hung from a hook, such as, but not limited to an IV stand. Hanging the first reservoir 302 may allow the first reservoir 302 to be positioned above the level of an endoscope cart which may enable the user to see the fluid 306 level at any time. This may help the clinician avoid running out of fluid during a procedure. Additionally, elevating the reservoir may eliminate the need for the clinician to bend or stoop during setup of the system 300 and/or to change the first reservoir 302. In some cases, head pressure generated from elevating the first reservoir 302 may enable rapid priming of the irrigation circuit (and/or lens wash circuit if so connected) which may save time during setup. It is further contemplated that hanging the first reservoir 302 from a hook or IV stand may allow the first reservoir 302 to be positioned away from expensive capital equipment thus reducing or eliminating the potential for fluid running or flowing inadvertently onto the capital equipment and causing damage or destruction.

The first reservoir 302 may be connected in fluid communication with a lumen of the upstream irrigation supply tube 328. The upstream irrigation supply tube 328 extends from a second end region 322 external to the container 304 and positioned within a pump head 324 of the peristaltic irrigation pump 315 to a first end 320. The first end 320 of the upstream irrigation supply tube 328 is coupled to the spike port adaptor 310 which in turn is configured to extend through a lumen of the port 308b and pierce a seal within the lumen of the port 308b to fluidly couple the interior of the container 304 with the lumen of the upstream irrigation supply tube 328. The second end of the upstream irrigation supply tube 328 is configured to be fluidly coupled with an irrigation lumen of the endoscope 100. When irrigation water is required, fluid is pumped from the first container 304 by operating the irrigation pump 315, such as by depressing a footswitch (not shown), and flows from the first reservoir 302, through the upstream irrigation supply tubing 328 and a branched connector 350, through the downstream irrigation supply tubing 255c, through the irrigation connection 293, through the irrigation feed line 255b in the umbilical 260, and down the irrigation supply line 255a in the shaft 100a of the endoscope to the distal tip 100c.

The downstream irrigation supply tubing 255c may include a loaded check valve or flow control valve 326 positioned in line with the downstream irrigation supply tubing 255c. The flow control valve 326 may prevent the unintentional flow of fluid from the first container 304 to the endoscope 100. In some cases, the flow control valve 326 may be configured to open when the pressure within the downstream irrigation supply line 255c reaches a predetermined minimum pressure. It is contemplated that the predetermined minimum pressure may be greater than the head pressure created by the height differential between the first reservoir 302 and the irrigation pump 315. The flow control valve 326 may also prevent fluid from leaking from the downstream irrigation supply tube 255c when the endoscope 100 is changed between patients and the tubing set connector is separated from the endoscope water port.

In some embodiments, the irrigation pump 315 may be omitted. For example, the reservoir 302 may be inserted into a compression sleeve. When irrigation fluid is desired, the compression sleeve may be activated to exert pressure on an outer surface of the reservoir 302 and to provide the required pressure to perform irrigation at the distal end of the endoscope 100. In another embodiment, the reservoir 302 may be inserted into a compression sleeve which applies constant pressure to the reservoir 302 with a flow switch positioned along irrigation supply tubing 328 to provide binary control of irrigation flow.

The second reservoir 330 may further include one or more ports 340, such as, but not limited to a spike port or a septum port, extending from and in selective fluid communication with an interior of the second container 332. The port 340 may be formed as a monolithic structure with the second container 332. The port 340 may be a generally tubular structure with the port 340 defining a lumen extending therethrough. The lumen of the port 340 may be configured to selectively fluidly couple the interior of the second container 332 with another component, such as, but not limited to, fluid/water/gas supply tube(s). In some cases, the port 340 may be configured to be coupled to the gas and lens wash supply tubing 336, 338. In some embodiments, the port 340 may be positioned adjacent to a bottom end 342 of the second reservoir 330. However, this is not required. The port 340 may be positioned in other locations, as desired. If the port 340 is positioned at a location other than the bottom end 342 of the second container 332, a dip tube or tube extension may be required (e.g., coupled to the lens wash supply tubing 338) to access the fluid at the bottom of the second container 332. While the second reservoir 330 is illustrated as including one port 340, the second reservoir 330 may include more than one port, as desired.

While not explicitly shown, the port 340 may include a removable cap or seal configured to form a fluid tight seal with the port 340. The removable cap or seal may help to maintain the sterility of the port 340. The removable cap or seal may be coupled to a free end of the port 340 using a number of different techniques. For example, the cap or seal may be coupled to the port 340 using a threaded engagement, a friction fit, a snap fit, etc., or may be fixedly coupled using a number of techniques such as adhesive or solvent bonding. In other instances, the cap or seal may be removed through a twisting motion configured to break the cap or seal from the port 340. Once the cap or seal has been removed, the port 340 may be pierced with a spike tip or spike port adaptor (not explicitly shown) that is coupled to the gas and lens wash supply tubing 336, 338. For example, in addition to the removable cap or seal, the port 340 may include an internal seal disposed within a lumen of the port 340 that may be punctured or pierced by the spike port adaptor. The internal seal may be configured to prevent fluid 334 from leaking from the second container 332 prior to the spike port adaptor being inserted into the port 340. In some embodiments, the internal seal may be self-scaling such that upon removal of the spike port adaptor fluid is prevented from leaking from the port 340. The outer surface of the spike port adaptor may form an interference fit with the inner surface of the port 340. The fit and/or coupling between the spike port adaptor and the port 340 may be sufficient to remain in place when the gas and fluid supply tubing 336, 338 and/or other tubing sets are coupled to the spike port adaptor. It is contemplated that the spike port adaptor may be inserted into the port 340 utilizing universally used aseptic techniques such as those used with IV fluid bags. This may help reduce infection risk by maintaining sterile components, not introducing contaminants into the fluid 334, etc. It is further contemplated that additives may be added to the fluid 334 using similar aseptic techniques via the port 340, if so desired. In some cases, other coupling mechanisms may be used as desired to couple the gas and lens wash supply tubing 336, 338 to the port 340. Some illustrative coupling mechanisms may include, but are not limited to, threaded engagements, snap fits, friction fits, quick connect style couplers, etc., or may be fixedly coupled using a number of techniques such as adhesive or solvent bonding.

The gas supply tubing 336 extends from a second end external to the second container 332 to the port 340. The gas supply tubing 336 may extend into the interior of the second container 332 and terminate within a reservoir gap (e.g., above the level of the fluid 334). However, in some cases, the gas supply tubing 336 may terminate within the fluid 334. A lumen extends through the gas supply tubing 336 for receiving a flow of air and/or gas therethrough. The lumen of the gas supply tubing 336 may be in operative fluid communication with a top portion of the interior of the second container 332. The lens wash supply tubing 338 extends from a second end external to the second reservoir 330 to a first end in fluid communication with a bottom portion 342 of the second container 332. In some embodiments, the lens wash supply tubing 338 may terminate at the port 340. A lumen extends through the lens wash supply tubing 338 for receiving a flow of fluid therethrough. The lumen of the lens wash supply 338 is in selective operative fluid communication with a bottom portion 342 of the second container 332. In the illustrated embodiment, the gas supply tubing 336 and the lens wash supply tubing 338 may couple to the second container 332 through a single or common opening (e.g., port 340). For example, the gas supply tubing 336 and the lens wash supply tubing 338 may be coaxially arranged. However, this is not required. In some cases, the gas supply tubing 336 and the lens wash supply tubing 338 may extend in a side by side arrangement or may be separately connected to the second container 332 in different locations.

The second container 332 may further include a first fluid inlet 344 and a second fluid inlet 346. While the first and second fluid inlets 344, 346 are illustrated as being adjacent to or extending from a top portion 348 of the second container 332, the first and/or second fluid inlets 344, 346 may be positioned at other locations about the second container 332, as desired. In some embodiments, the first and/or second fluid inlets 344, 346 may be tubular members formed as a single monolithic structure with the second container 332. In other embodiments, the first and/or second fluid inlets 344, 346 may include tubular components releasably coupled to ports (similar in form and function to port 340) formed in or with the container 332.

The first fluid inlet 344 may be in selective fluid communication with the first reservoir 302. For example, a branched connector 350 may be positioned in-line with the upstream irrigation tubing 328. In some embodiments, the branched connector 350 may be a “Y” connector or a “T” connector having an inlet leg 356 defining a first fluid inlet, a first outlet leg 352 defining a first fluid outlet, and a second outlet leg 354 defining a second fluid outlet. However, it is contemplated that the branched connector 350 may include more than one fluid inlet and fewer than two or more than two fluid outlets, if so desired.

The branched connector 350 may be positioned in-line with the upstream irrigation tubing 328 such that the inlet leg 356 and the first outlet leg 352 are fluidly coupled with the lumen of the upstream irrigation tubing 328. Fluid may flow from the first reservoir 302, through the upstream irrigation tubing 328, through the branched connector 350 and again through the upstream irrigation tubing 328. The branched connector 350 may be positioned such that the inlet leg 356 is upstream of the outlet legs 352, 354 relative to a flow of irrigation fluid. In some embodiments, the branched connector 350 and the spike port adaptor 310 may be molded or formed as a single monolithic structure. It is contemplated that this may reduce connection points in the fluid circuit. In such an instance, the first end 320 of the irrigation supply tubing 328 may be fluidly coupled to the first outlet leg 352 of the branched connector 350.

The second outlet leg 354 may be fluidly coupled to the first fluid inlet 344 of the second reservoir 330. A flow control mechanism, such as, but not limited to, a one-way valve 358 may be positioned between the second fluid outlet of the second outlet leg 354 and the first fluid inlet 344 of the second reservoir 330 to selectively fluidly couple the second container 332 with the first container 304. The one-way valve 358 may be configured to be opened to allow fluid to selectively pass from the first reservoir 302 to the second reservoir 330 while preventing fluid (e.g., gas, water, or other fluid) from exiting the second container 332 and entering the irrigation supply tubing 328 and/or the first container 304. In some embodiments, the one-way valve 358 may be replaced with a clamp which may compress the first fluid inlet 344 to selectively fluidly isolate the second container 332 from the first container 304 and removed to selectively couple the second container 332 with the first container 304. In yet other embodiments, the one-way valve 358 may be replaced with a spring-loaded valve, a stopcock, or other two-way valve. When it is desired to add fluid to the second reservoir 330 from the first reservoir 302, the one-way valve 358 (or other flow control mechanism) may be opened or released. Fluid may then be at least partially diverted from the irrigation supply tubing 328 through the second outlet leg 354 of the branched connector 350 and into the second container 332 along flow path 360. Fluid may be added to the second container 332 while the irrigation pump 315 is running or while the irrigation pump 315 is idle, as desired.

The second fluid inlet (or gas supply tube) 346 of the second container 332 may be an alternative gas supply tubing configured to be coupled to an alternative gas supply (e.g., CO₂ hospital house gas source). The second fluid inlet 346 may extend from a second end external to the second container 332 to a first end coupled to the second container 332. The alternative gas supply may be used to pressurize the second container 332 to supply lens wash to the endoscope 100 and/or to provide insufflation. A lumen extends through the second fluid inlet 346 for receiving a flow of gas therethrough. The lumen of the second fluid inlet 346 is in operative fluid communication with a top portion of the second container 332. The flow of the CO₂ through the system 300 may be similar to that described above. For example, in the neutral state, CO₂ gas flows through the second fluid inlet 346 into the second container 332, up the gas supply tubing 336 to the connector portion 265, up the gas feed line 240b in the umbilical 260, and is vented through the gas/water valve 140 to atmosphere. In the first position, the user closes off the vent hole in the gas/water valve 140, and the CO₂ gas is flowed through the second fluid inlet 346 into the second container 332, up the gas supply tubing 336 to the connector portion 265, through the gas/water valve to the gas supply line 240a in the endoscope shaft 100a and out the gas/lens wash nozzle 220 at the distal tip 100c. In the second position, the user depresses the valve 140 to the bottom of the valve well 135, keeping the vent hole in the gas/water valve closed off. The second position blocks the CO₂ gas supply to both atmosphere and the gas supply line 240a in the endoscope 100, and opens up the gas/water valve 140 to allow lens wash water to pass through to the lens wash supply line 245a in the endoscope shaft 100a and out the gas/lens wash nozzle 220 at the distal tip 100c. Gas (pressure) in the second reservoir 330 is maintained by delivering gas through the second fluid inlet 346. It is contemplated that the one-way valve 358 is in the closed configuration during delivery of the CO₂ gas to allow the container 332 to pressurize. In some instances, the one-way valve 358 may be configured to close without user intervention in response to the delivery of CO₂ to the second container 332. In some embodiments, the system 300 may include a branched connector (such as, but not limited to a “Y” or “T” connector) at the second fluid inlet 346 to allow either air or CO₂ to be used for pressurization or insufflation. It is further contemplated that the second fluid inlet 346 may include a pressure relief valve 362, such as, but not limited to, a 3-way stopcock, a clamp, or a spring-loaded valve, to vent pressure within the second container 332 and/or to block a flow of pressurized gas to the second container 332 during refilling of the second container 332, during procedure change-overs, and/or during equipment change-overs.

It is contemplated that the use of a flexible bag in place of a rigid bottle for the second reservoir 330 may reduce or eliminate the risk of air leaking from bottle and cap connections. This may eliminate the need for clinicians to attempt to remedy the leak by adjusting the cap and bottle assemblies or from discarding a cap and/or bottle if the leak cannot be remedied.

As the pressurized second container 332 is fluidly isolated from the first container 304 when the one-way valve 358 is closed, it is contemplated that the clinician may replace the first reservoir 302 with a new (full) reservoir without losing patient insufflation. Loss of patient insufflation may result in a loss of position of the endoscope 100 within the body. In current one or two bottle systems, it may not be possible to replace the water reservoirs without loss of patient insufflation.

If there is a need to replace the first reservoir 302 with a new full bag, for example when the first reservoir 302 is empty or near empty, the user may hang the new bag near the first reservoir 302 to be replaced. The user may then disengage the spike port adaptor 310 from the port 308b and insert the spike port adaptor 310 into a port of the new bag. This may be performed without requiring the clinician to bend or stoop to access the first reservoir 302. The port 308b may self-seal to prevent fluid leaks from the first reservoir 302 being replaced. This method of replacing the first reservoir 302 may have a lower risk of introducing contaminants into the systems relative to traditional bottle systems. For example, the change out method described herein may allow the first reservoir 302 to be changed out without having tubing dangling from a cap (as in a bottle system). Further, the system 300 may remain largely closed as the first reservoir 302 is changed out.

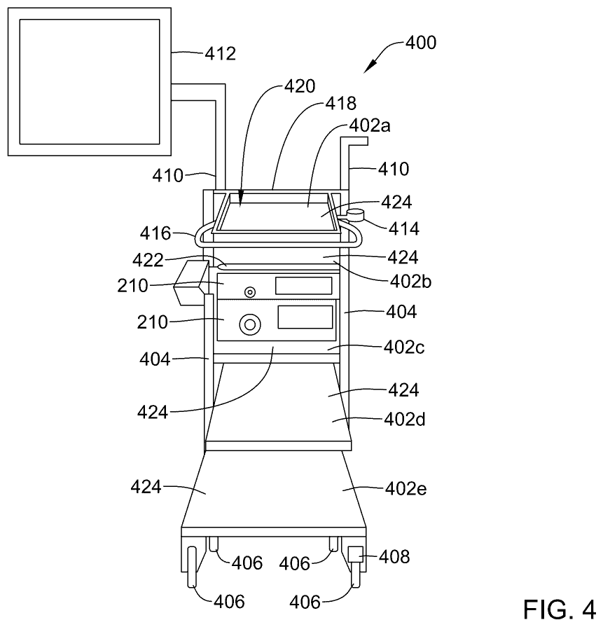

FIG. 4 is a schematic perspective view of an illustrative cart 400 that may be used to hold or mount capital equipment during an endoscopic procedure. The illustrative cart 400 may include a plurality of shelves 402a-e movably or fixedly secured to a frame 404. The cart 400 may include any number of shelves 402a-e desired. The shelves 402a-e may be configured to store equipment thereon. For example, one or more processing units 210 may be positioned one or more of the shelves 402a-e. The shelves 402a-e may include differing structures to accommodate different components of the endoscope system 200. For example, some shelves 402c-e may be generally planar to allow equipment to be easily positioned thereon and/or removed therefrom. Other shelves 402a may include a raised perimeter 418 defining an inner cavity or recess 420. This may help prevent items from falling off the shelf 402. In yet other examples, shelves 402b may include a raised lip 422 along one or more edges thereof to facilitate gripping of the shelf 402b. Each shelf 402a-e may include an upper surface 424 upon which equipment may be positioned.

The cart 400 may further include a plurality of wheels 406 to facilitate positioning of the cart 400 within the procedure suite. In some cases, one or more of the wheels 406 may include a locking mechanism 408 configured to prevent movement of the wheel 406 and/or cart 400. One or more arms or poles 410 may extend from the frame 404. The poles 410 may be configured to mount various components thereto, such as, but not limited to, a display unit 412. In some cases, the poles 410 may include features configured to receive tube sets, endoscope handles, and the like. Alternatively, or additionally, mounting features 414 for receiving tube sets, endoscope handle, and the like may be mounted to one or more of the shelves 402a-e and/or the frame 404. The cart 400 may include one or more handles, push bars or holding bars 416. The bars 416 may be formed as a single monolithic structure with or coupled to one of the shelves 402a-c.

Alternatively, or additionally, the bars 416 may be formed as a single monolithic structure with or coupled to the frame 404. It should be understood that the cart 400 may include additional features not expressly described herein. Further the shelves 402a-c, frame 404, poles 410, and/or other features may be arranged in any manner desired.

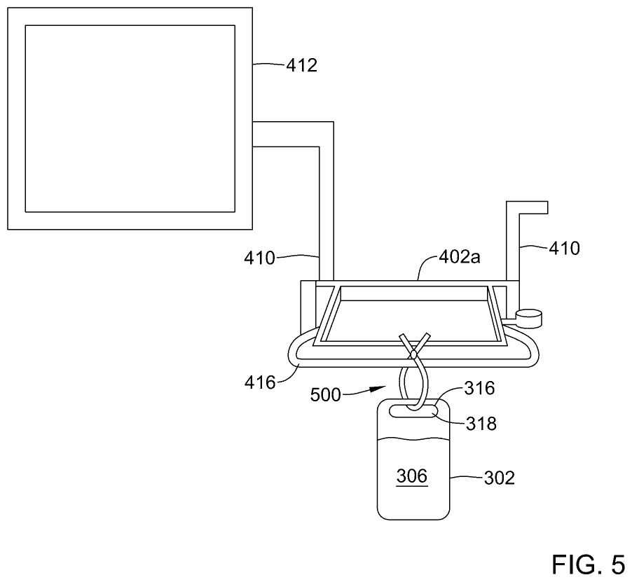

As described herein, it may be desirable to mount one or more of the reservoirs 302, 330 from the cart 400 or to equipment coupled thereto. FIG. 5 is a perspective view of a portion of the cart 400 including an upper shelf 402a of the endoscope cart 400 having a push bar 416. A reservoir 302 may be releasably secured to the bar 416 with a coupling member 500. The coupling member 500 may be configured to be wrapped, tied, or otherwise secured about a portion of the perimeter of the bar 416. It is contemplated that the coupling member 500 may be coupled with other parts of the cart 400, such as, but not limited to, an arm 410 or the frame 404. The coupling member 500 may extend through or about a handle 316 or other opening or hanging means of the reservoir 302 before being coupled with the bar 416. It is contemplated that the reservoir 302 may be secured to the bar 416 anywhere along the length of the bar 416. Further, the coupling member 500 may be secured such that the coupling member 500 may be moved along the bar 416 to adjust a position of the reservoir 302 if so desired. Securing the reservoir 302 to the bar 416 may allow the reservoir 302 to be positioned in a location that allows the clinician to see an amount of fluid 306 that remains within the reservoir 302 while maintaining the reservoir 302 in a location that does not interfere with the procedure.

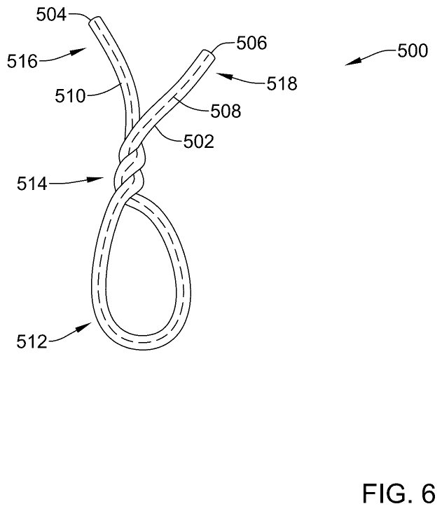

FIG. 6 is a perspective view of the illustrative coupling member 500. The coupling member 500 may include an elongate body 502 extending from a first end 504 to a second end 506. The elongate body 502 may include a flexible and/or deformable wire 508 extending from the first end 504 to the second end 506. The wire 508 may be configured to be moved into a plurality of different configurations. The deformable wire 508 may be formed from a material including metals, such as, but not limited to, aluminum, copper, which allows the wire 508 to be repeatedly bent and unbent (e.g., plastically deformed) without fracturing. When moved into a new configuration, the wire 508 may hold its shape without any additional fixation means. The wire 508 may be coated with a coating 510. The coating 510 may be formed from a polymeric material, such as, but not limited to, silicone, or the like. It is contemplated that the coating 510 may increase a surface area of the coupling member 500 (relative to using only the wire 508) to improve the gripability and maneuverability of the coupling member 500. In some cases, the coating 510 may increase the friction between the coupling member 500 and the bar 416 such that the coupling member 500 better grips the bar 416.

The coupling member 500 may be movable between an elongated configuration (not explicitly shown) where the elongate body 502 does not cross-over itself to a twisted configuration configured to form a loop 512. To hang the reservoir 302 from the bar 416, the coupling member 500 may first be in an elongated configuration. The first end 504 (or second end 506) of the elongate body 502 may be inserted through the opening 318 of the handle 316 of the reservoir 302. The first end 504 may then be positioned on a first side of the bar 416 and the second end 506 positioned on a second side of the bar 416 with the each of the first and second ends 504, 506 above the bar 416 (or at an opposing side from the handle 316 of the reservoir 302). One or both of the first or second ends 504, 506 may be twisted or wound relative to the other to form a twisted region 514. The twisted region 514 may be axially offset from the first end 504 and the second end 506 such that in the twisted configuration the coupling member 500 includes a loop 512, the twisted region 514, a first free end region 516, and a second free end region 518. A length of the first free end region 516 and/or a length of the second free end region 518 may vary depending on the location of the twisted region 514. Further, the diameter, or cross-sectional dimension of the loop 512 may vary depending on the location of the twisted region 514. For example, the larger the diameter of the loop 512, the shorter the length of the first and/or second free end regions 516, 518 may be. The twisted region 514 may be positioned such that the bar 416 and a portion of the handle 316 of the reservoir 302 are disposed within an interior of the loop 512. If it is desired to remove the coupling member 500 from the bar 416 (or other portion of the cart 400), the twisting action of the first and/or second ends 504, 506 may be reversed to move the coupling member 500 to the elongated configuration. Once the twisted region 514 has been removed, the coupling member 500 may be disengaged from the bar 416 and/or the handle 316.

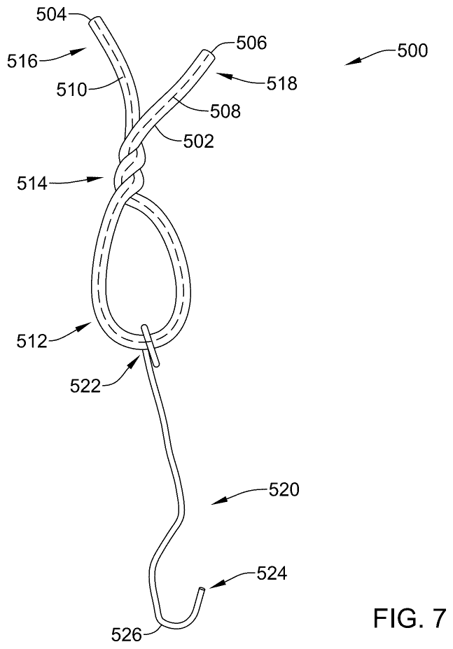

In some embodiments, the coupling member 500 may further include a hook or hook-like member 520 releasably coupled thereto. FIG. 7 is a perspective view of the illustrative coupling member 500 of FIG. 6 with an optional hook 520. The hook 520 may extend from a first end region 522 to a second end region 524. The first end region 522 may be configured to be secured or coupled to the loop 512 of the coupling member 500. In some examples, the first end region 522 may be wound around the elongate body 502 of the coupling member 500 within the loop 512. However, the hook 520 may be coupled with the coupling member 500 using other techniques, such as, but not limited to, clamps, clips, adhesives, or the like. In yet other examples, the first end region 522 may have a curved region that hooks over the elongate body 502 of the coupling member 500. The second end region 524 may include a curved region 526 configured to receive the handle 316 of the reservoir 302. The curved region 526 may have a generally “U” or “J” shape such that the handle 316 may rest within an interior of the curved region 526. The hook 520 may be formed from a variety of different materials including metallic materials, such as, but not limited to, aluminum, stainless steel, metal alloys, and the like, polymeric materials, such as, but not limited to, acrylonitrile butadiene styrene (ABS), polyimide, polyamide-imide (PAI), polyamide, polyether ether ketone (PEEK), or the like, or from polymer materials reinforced with glass fiber, ground mica, nanoclay, graphene, talc, kaolin, wollastonite, and the like.

When the hook 520 is used to hold or receive the reservoir 302, the coupling member 500 may be affixed to the bar 416 prior to coupling the hook 520 and/or the reservoir thereto. For example, the coupling member 500 may be secured about the bar 416 and then the hook 520 may be coupled thereto. Alternatively, the hook 520 may be coupled to the coupling member 500 prior to securing the coupling member 500 to the bar 416. Once the hook 520 is coupled with the coupling member 500, the reservoir 302 may be hung from the hook 520.

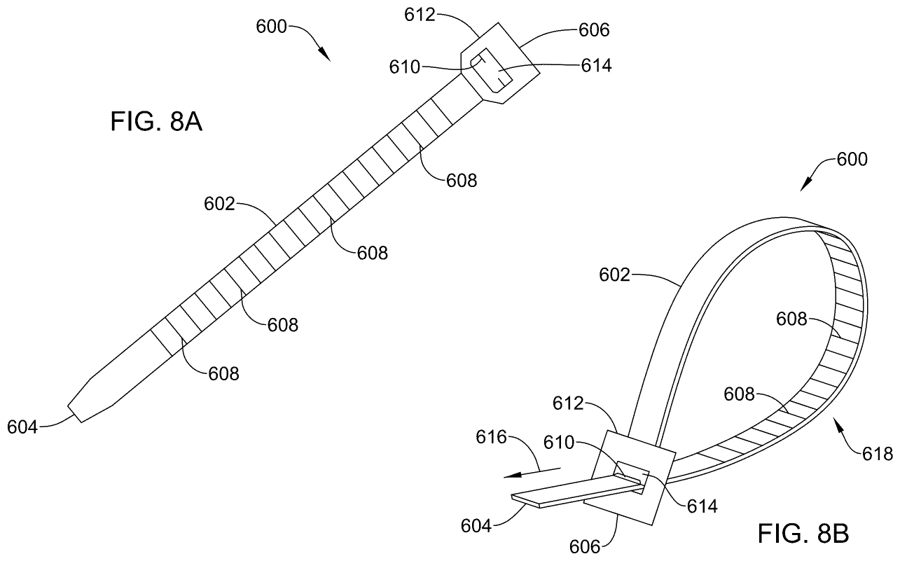

FIG. 8A is a perspective view of another illustrative coupling member 600 in a first elongated configuration and FIG. 8B is a perspective view of the illustrative coupling member 600 of FIG. 8A in a second looped configuration. The coupling member 600 may include an elongate body 602 extending from a first end 604 to a second end 606. A first side of the elongate body 602 may include a plurality of teeth 608. The teeth 608 may be raised regions configured to engage a pawl 610 disposed within an opening 614 of an insertion head 612. The first end 604 of the elongate body 602 may be configured to be inserted into the opening 614 of the insertion head 612. The pawl 610 may allow for the first end 604 of the elongate body 602 to be moved through the opening 614 in a first direction, as shown at arrow 616 in FIG. 8B. However, once the pawl 610 engages at least a first tooth of the plurality of teeth 608, movement of the first end 604 in a second direction, opposite the first direction, may be precluded. The pawl 610 may engage one or more of the teeth 608 to maintain the coupling member 600 in a looped configuration, as shown in FIG. 8B.

The coupling member 600 may be movable between the first generally elongated configuration (FIG. 8A) and the looped configuration (FIG. 8B) to form a loop 618. To hang the reservoir 302 from the bar 416, the coupling member 600 may first be in the elongated configuration. The first end 604 (or second end 606) of the elongate body 602 may be inserted through the opening 318 of the handle 316 of the reservoir 302. The first end 604 may then be positioned on a first side of the bar 416 and the second end 606 positioned on a second side of the bar 416 with the each of the first and second ends 604, 606 above the bar 416 (or at an opposing side from the handle 316 of the reservoir 302). The first end 604 may then be inserted through the opening 614 of the insertion head 612 until the at least one tooth 608 engages the pawl 610. The pawl 610 may engage one or more teeth 608 to hold the coupling member 600 in the looped configuration. In the looped configuration, the first end 604 of the elongate body 602 may extend from the insertion head 612. The length of the elongate body 602 extending from the insertion head 612 may be trimmed or removed or left untouched. The diameter, or cross-sectional dimension of the loop 618 may vary depending on how far the first end 604 is pulled through the insertion head 612. For example, the larger the diameter of the loop 618, the shorter the length of the elongate body 602 extending from the insertion head 612 may be. If it is desired to remove the coupling member 600 from the bar 416 (or other portion of the cart 400), the elongate body 602 may be cut or severed. In some cases, the pawl 610 may include a release mechanism configured to disengage the pawl 610 from the teeth 608 to allow the first end 604 to be retracted through the pawl 610 without mechanical damage to the coupling member 600. Once the first end 604 has been retracted from the insertion head 612 or the coupling member 600 has been cut, the coupling member 600 may be disengaged from the bar 416 and/or the handle 316.

The coupling member 600 may be formed from a variety of different materials including metallic materials, such as, but not limited to, aluminum, stainless steel, metal alloys, and the like, polymeric materials, such as, but not limited to, acrylonitrile butadiene styrene (ABS), polyimide, polyamide-imide (PAI), polyamide, polyether ether ketone (PEEK), or the like, or from polymer materials reinforced with glass fiber, ground mica, nanoclay, graphene, talc, kaolin, wollastonite, and the like.

In some embodiments, the coupling member 600 may further include a hook or hook-like member releasably coupled thereto. The hook may be similar in form and function to the hook 520 described with respect to FIG. 7. Generally, the hook may extend from a first end region to a second end region. The first end region may be configured to be secured or coupled to the loop 618 of the coupling member 600. The second end region may include a curved region configured to receive the handle 316 of the reservoir 302. The curved region may have a generally “U” or “J” shape such that the handle 316 may rest within an interior of the curved region. The hook may be formed from a variety of different materials including metallic materials, such as, but not limited to, aluminum, stainless steel, metal alloys, and the like, polymeric materials, such as, but not limited to, acrylonitrile butadiene styrene (ABS), polyimide, polyamide-imide (PAI), polyamide, polyether ether ketone (PEEK), or the like, or from polymer materials reinforced with glass fiber, ground mica, nanoclay, graphene, talc, kaolin, wollastonite, and the like.

When the hook is used to hold or receive the reservoir 302, the coupling member 600 may be affixed to the bar 416 prior to coupling the hook and/or the reservoir thereto. For example, the coupling member 600 may be secured about the bar 416 and then the hook may be coupled thereto. Alternatively, the hook may be coupled to the coupling member 600 prior to securing the coupling member 600 to the bar 416. Once the hook is coupled with the coupling member 600, the reservoir 302 may be hung from the hook.

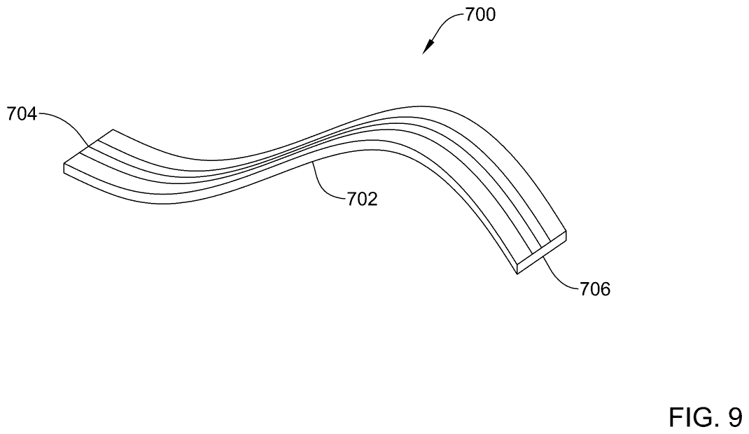

FIG. 9 is a perspective view of another illustrative coupling member 700 in a first elongated configuration. The coupling member 700 may include an elongate body 702 extending from a first end 704 to a second end 706. The elongate body 702 may be formed from a flexible material, such as, but not limited to, natural rubber, ethylene propylene diene monomer (EPM, EPDM), synthetic polyisoprene, silicone, rubber (or stretchable synthetic, such as spandex) cores that are bound or wrapped in polyester, cotton, nylon or a blend of fiber threads, or the like. In some examples, the elongate body 702 may include a coating or otherwise be formed from a material configured to increase the friction between the elongate body 702 and the bar 416.

The coupling member 700 may be movable between the first generally elongated configuration (FIG. 9) and a looped configuration (not explicitly shown). To hang the reservoir 302 from the bar 416, the coupling member 700 may first be in an elongated configuration. The first end 704 (or second end 706) of the elongate body 702 may be inserted through the opening 318 of the handle 316 of the reservoir 302. The first end 704 may then be positioned on a first side of the bar 416 and the second end 706 positioned on a second side of the bar 416 with the each of the first and second ends 704, 706 above the bar 416 (or at an opposing side from the handle 316 of the reservoir 302). The free ends of the elongate body 702 may then be knotted or formed into a knot to form a looped region. The knot may be axially offset from the first end 704 and the second end 706 such that in the looped configuration the coupling member 700 includes a loop, the knot, a first free end region, and a second free end region. A length of the first free end region and/or a length of the second free end region may vary depending on the location of the knot. Further, the diameter, or cross-sectional dimension of the loop may vary depending on the location of the knot. For example, the larger the diameter of the loop, the shorter the length of the first and/or second free end regions may be. The knot may be positioned such that the bar 416 and a portion of the handle 316 of the reservoir 302 are disposed within an interior of the loop. If it is desired to remove the coupling member 700 from the bar 416 (or other portion of the cart 400), the knot may be untied to move the coupling member 700 to the elongated configuration. Once the knot has been removed, the coupling member 700 may be disengaged from the bar 416 and/or the handle 316. Alternatively, the coupling member 700 may be cut or severed for removal from the bar 416.

In some embodiments, the coupling member 700 may further include a hook or hook-like member releasably coupled thereto. The hook may be similar in form and function to the hook 520 described with respect to FIG. 7. Generally, the hook may extend from a first end region to a second end region. The first end region may be configured to be secured or coupled to the loop of the coupling member 700. The second end region may include a curved region configured to receive the handle 316 of the reservoir 302. The curved region may have a generally “U” or “J” shape such that the handle 316 may rest within an interior of the curved region. The hook may be formed from a variety of different materials including metallic materials, such as, but not limited to, aluminum, stainless steel, metal alloys, and the like, polymeric materials, such as, but not limited to, acrylonitrile butadiene styrene (ABS), polyimide, polyamide-imide (PAI), polyamide, polyether ether ketone (PEEK), or the like, or from polymer materials reinforced with glass fiber, ground mica, nanoclay, graphene, talc, kaolin, wollastonite, and the like.

When the hook is used to hold or receive the reservoir 302, the coupling member 700 may be affixed to the bar 416 prior to coupling the hook and/or the reservoir thereto. For example, the coupling member 700 may be secured about the bar 416 and then the hook may be coupled thereto. Alternatively, the hook may be coupled to the coupling member 700 prior to securing the coupling member 700 to the bar 416. Once the hook is coupled with the coupling member 700, the reservoir 302 may be hung from the hook.

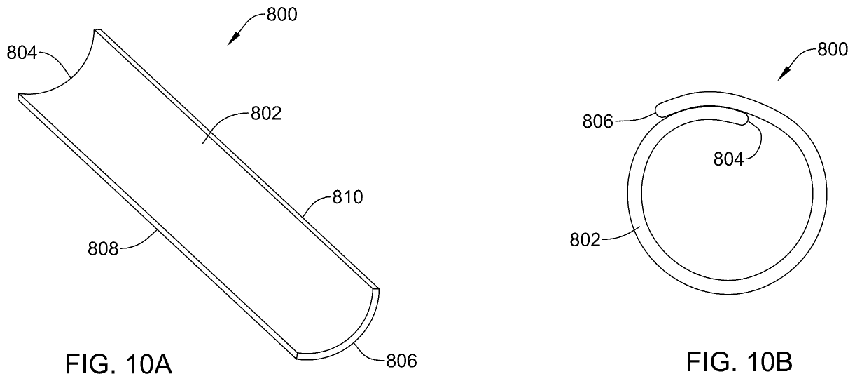

FIG. 10A is a perspective view of another illustrative coupling member 800 in a first elongated configuration. FIG. 10B is an end view of the illustrative coupling member 800 of FIG. 10A in a second looped configuration. The coupling member 800 may include an elongate body 802 extending from a first end 804 to a second end 806. The elongate body 802 may further include a first lateral side 808 and a second lateral side 810. In the elongated configuration, the elongate body 802 may have a generally curved configuration between the first lateral side 808 and the second lateral side 810. The coupling member 800 may be formed from a bistable spring. For example, the coupling member 800 may maintain either the elongated configuration of FIG. 10A or the curled or looped configuration of FIG. 10B when the coupling member 800 is left undisturbed. Tapping the first or second end 804, 806 of the elongate body 802 on another surface when the coupling member 800 is in the elongated configuration may cause the elongate body 802 to flatten (between the lateral sides 808, 810) and curl upon itself, as shown in FIG. 10B with the first and second ends 804, 806 coming towards one another. In some cases, one of the first or second ends 804, 806 may overlap the other. In other cases, the first and second ends 804, 806 may be spaced a distance from one another. In yet other cases, the first and second ends 804, 806 may just contact one another. The elongate body 802 may remain in the looped or curled configuration until a force is applied to the elongate body 802. It is contemplated that to move the elongate body 802 to the elongated configuration, the first and second ends 804, 806 may be pulled away from one another and a force exerted on a central region of the elongate body 802 to move the elongate body 802 to the curved configuration (e.g., curved between the first and second lateral sides 808, 810).

The coupling member 800 may be a composite material that is a combination of metal, silicone, and/or plastic that allows the coupling member 800 to maintain a straight elongated configuration and a curled or looped configuration. In some examples, the elongate body 802 may include a base material formed from a spring metal, such as, but not limited to, steel. The base material may be optionally coated with polymeric material, such as, but not limited to, silicone, such that the coupling member 800 may more securely grip the bar 416.

As described herein, the coupling member 800 may be movable between the first generally elongated configuration (FIG. 10A) and a looped or curled configuration (FIG. 10B). To hang the reservoir 302 from the bar 416, the coupling member 800 may first be in an elongated configuration. The first end 804 (or second end 806) of the elongate body 802 may be inserted through the opening 318 of the handle 316 of the reservoir 302. The first end 804 may then be positioned on a first side of the bar 416. The first or second end 804, 806 of the elongate body 802 may be tapped or pushed to cause the elongate body 802 to coil upon itself. As the elongate body 802 curls, the coupling member 800 may wrap around the bar 416 and the handle 316 of the reservoir 302 to secure the reservoir 302 to the bar 416. It in contemplated that in the curled or looped configuration the elongate body 802 closely contacts the bar 416 and/or the handle 316 such that the interior of the loop is filled or substantially filled with the bar 416 and/or handle 316. If it is desired to remove the coupling member 800 from the bar 416 (or other portion of the cart 400), the first and second ends 804, 806 may be moved away from one another and the elongate body 802 “snapped” into the elongated configuration. In some cases, the elongate body 802 may not be fully transitioned to the elongated configuration for removal. As the first and second ends 804, 806 are moved from one another, the coupling member 800 may be disengaged from the bar 416 and/or the handle 316.

In some embodiments, the coupling member 800 may further include a hook or hook-like member releasably coupled thereto. The hook may be similar in form and function to the hook 520 described with respect to FIG. 7. Generally, the hook may extend from a first end region to a second end region. The first end region may be configured to be secured or coupled to the loop of the coupling member 800. The second end region may include a curved region configured to receive the handle 316 of the reservoir 302. The curved region may have a generally “U” or “J” shape such that the handle 316 may rest within an interior of the curved region. The hook may be formed from a variety of different materials including metallic materials, such as, but not limited to, aluminum, stainless steel, metal alloys, and the like, polymeric materials, such as, but not limited to, acrylonitrile butadiene styrene (ABS), polyimide, polyamide-imide (PAI), polyamide, polyether ether ketone (PEEK), or the like, or from polymer materials reinforced with glass fiber, ground mica, nanoclay, graphene, talc, kaolin, wollastonite, and the like.

When the hook is used to hold or receive the reservoir 302, the coupling member 800 may be affixed to the bar 416 prior to coupling the hook and/or the reservoir thereto. For example, the coupling member 800 may be secured about the bar 416 and then the hook may be coupled thereto. Alternatively, the hook may be coupled to the coupling member 800 prior to securing the coupling member 800 to the bar 416. Once the hook is coupled with the coupling member 800, the reservoir 302 may be hung from the hook.

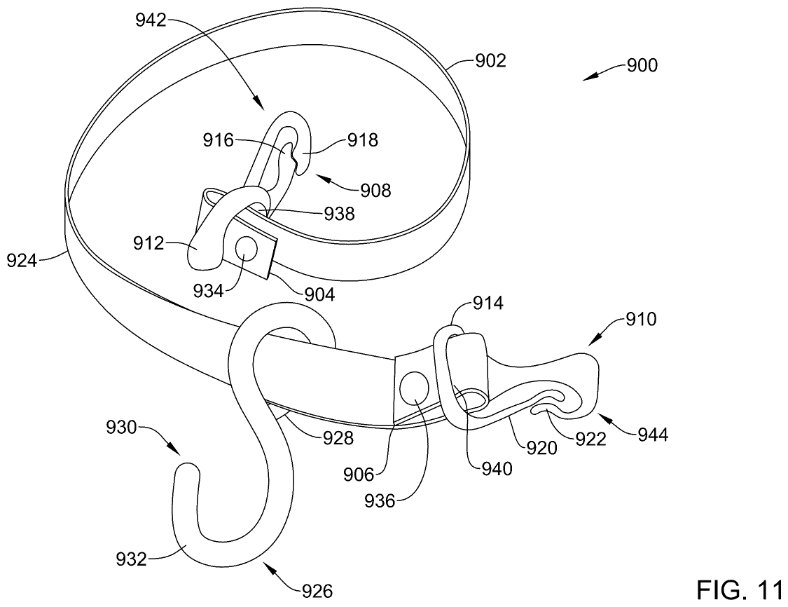

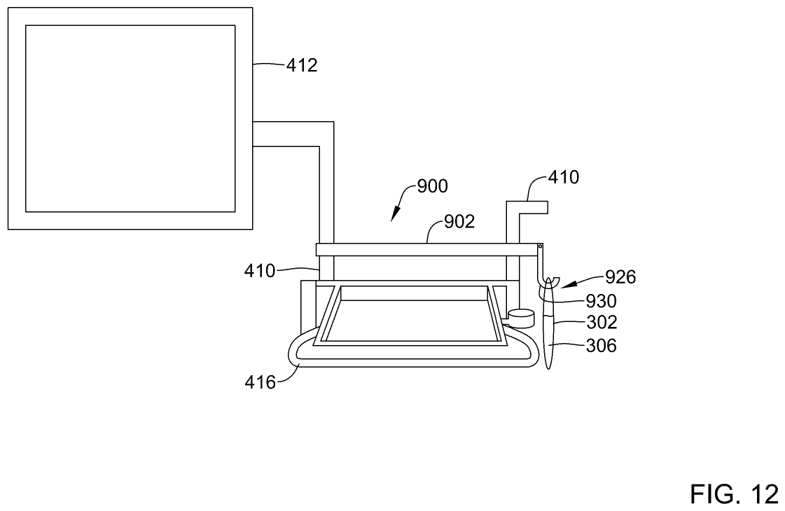

FIG. 11 is a perspective view of another illustrative coupling member 900. FIG. 12 is a partial front view of the illustrative cart 400 having the coupling member 900 secured thereto. The coupling member 900 may include an elongate body 902 extending from a first end 904 to a second end 906. In some embodiments, the elongate body 902 may be a flexible strap. The elongate body 902 may be formed from cotton, nylon, canvas, elastic, other fabrics, natural rubber, ethylene propylene diene monomer (EPM, EPDM), synthetic polyisoprene, silicone, rubber, flexible polymers, and the like.

The first end 904 of the elongate body 902 may be secured to a first buckle or clasp 908. The first buckle 908 may have a clasp end 942 and a securement end 912. The first end 904 may have a length of material that is looped through an opening or slot 938 of the securement end 912 of the buckle 908 such that first end 904 doubles back over a portion of the elongate body 902. The elongate body 902 may be secured to itself adjacent the first end 904 thereof using a coupling mechanism 934, such as, but not limited to, buttons, snaps, rivets, adhesives, fusible tape, fusible web, and the like. It is contemplated that the elongate body 902 may be fixedly or removably coupled with the first buckle 908. In the illustrated embodiments, the first buckle 908 may be a carabiner style clasp. For example, the clasp end 942 may include a movable gate 916 configured to engage a nose 918. The gate 916 may be depressed inwards to secure allow another component to be passed into an interior of the first buckle 908 while the nose 918 prevents the gate 916 from unintentional opening in a direction opposite the interior thereof. It is contemplated that the first buckle 908 may take other forms, as desired. Some illustrative buckles or clasps may include, but are not limited to, side release buckles, lobster clasps, magnetic clasps, S-hooks, spring rings, bayonet style locking mechanisms, and the like.

The second end 906 of the elongate body 902 may be secured to a second buckle or clasp 910. The second buckle 910 may have a clasp end 944 and a securement end 914. The second end 906 may have a length of material that is looped through an opening or slot 940 of the securement end 914 of the buckle 910 such that second end 906 doubles back over a portion of the elongate body 902. The elongate body 902 may be secured to itself adjacent the second end 906 thereof using a coupling mechanism 936, such as, but not limited to, buttons, snaps, rivets, adhesives, fusible tape, fusible web, and the like. It is contemplated that the elongate body 902 may be fixedly or removably coupled with the second buckle 910. In the illustrated embodiments, the second buckle 910 may be a carabiner style clasp. For example, the clasp end 944 may include a movable gate 920 configured to engage a nose 922. The gate 920 may be depressed inwards to secure allow another component to be passed into an interior of the second buckle 910 while the nose 922 prevents the gate 920 from unintentional opening in a direction opposite the interior thereof. It is contemplated that the second buckle 910 may take other forms, as desired. Some illustrative buckles or clasps may include, but are not limited to, side release buckles, lobster clasps, magnetic clasps, S-hooks, spring rings, bayonet style locking mechanisms, and the like. In some embodiments, at least one of the first or second buckles 908, 910 may be replaced with a ring or other structure configured to be received by the other of the first or second buckles 908, 910.

An intermediate length 924 of the elongate body 902 may extend between the first buckle 908 and the second buckle 910. A hook 926 may be movably secured to the intermediate length 924 of the elongate body 902. The hook 926 may extend from a first end region 928 to a second end region 930. The first end region 928 may be configured to be secured or coupled to the elongate body 902. In some examples, the first end region 928 may be wound or wrapped around the elongate body 902 of the coupling member 900. However, the hook 926 may be coupled with the coupling member 900 using other techniques, such as, but not limited to, clamps, clips, adhesives, or the like. In yet other examples, the first end region 928 may have a curved region that hooks over the elongate body 902 of the coupling member 900. The second end region 930 may include a curved region 932 configured to receive the handle 316 of the reservoir 302. The curved region 932 may have a generally “U” or “J” shape such that the handle 316 may rest within an interior of the curved region 932. The hook 926 may be formed from a variety of different materials including metallic materials, such as, but not limited to, aluminum, stainless steel, metal alloys, and the like, polymeric materials, such as, but not limited to, acrylonitrile butadiene styrene (ABS), polyimide, polyamide-imide (PAI), polyamide, polyether ether ketone (PEEK), or the like, or from polymer materials reinforced with glass fiber, ground mica, nanoclay, graphene, talc, kaolin, wollastonite, and the like.

To secure the coupling member 900 to the cart, the elongate body 902 may be wrapped around a portion of the cart 400. In FIG. 12, the coupling member 900 is shown as wrapped around arms 410 of the cart 400. However, the coupling member 900 may be wrapped around the frame 404, a shelf 402a-e, or the like. The first and second buckles 908, 910 may engage one another (e.g., may be clipped, clasped, or otherwise coupled) to form a loop. The hook 926 may be slidably disposed along the intermediate length 924 of the elongate body 902 to position the hook 926 in a desired location. In some cases, the elongate body 902 may include a slide, such as, but not limited to, a tri-glide slide configured to adjust a length of the elongate body 902. For example, it may be desirable for the elongate body 902 to form a taut loop about the cart 400. Once the hook 926 is in the desired position, the handle 316 of the reservoir 302 may be placed in the curved region 932 of the hook 926. It is contemplated that the hook 926 may be positioned at a location that allows a clinician a clear view of the fluid 306 within the reservoir 302. To remove the coupling member 900 from the cart 400, the first and second buckles 908, 910 may be released.

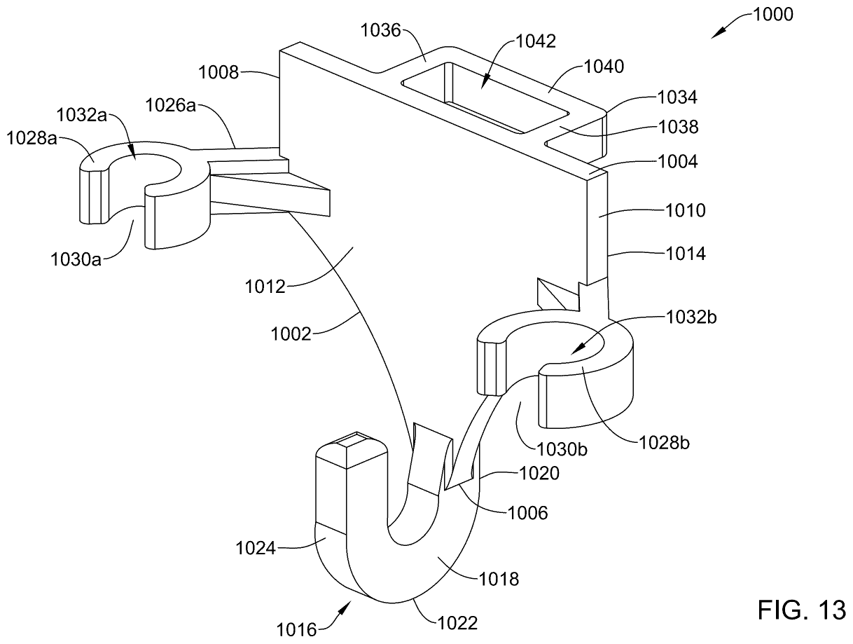

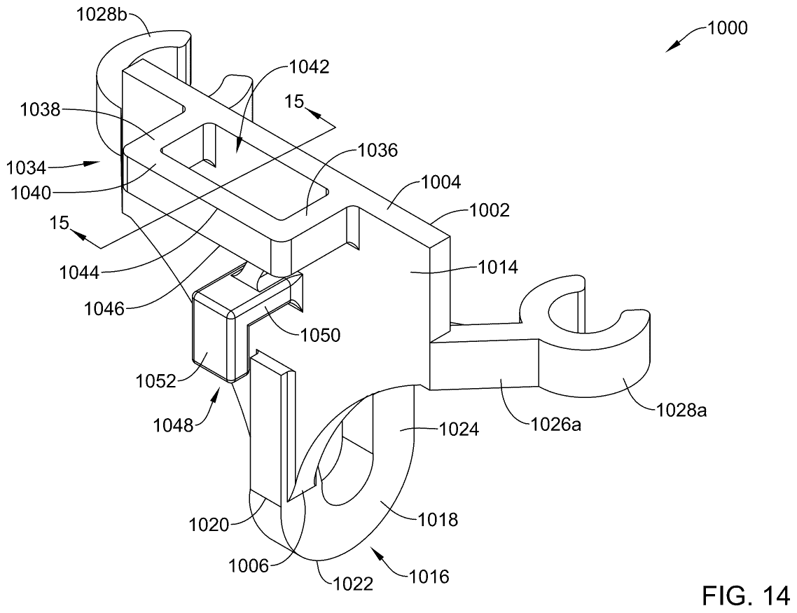

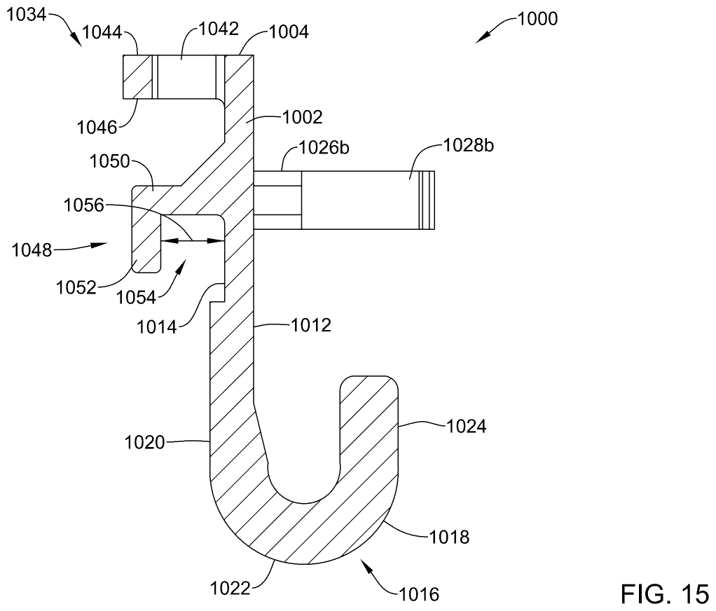

FIG. 13 is a front perspective view of another illustrative coupling member 1000. FIG. 14 is a rear perceptive view of the illustrating coupling member 1000 of FIG. 14. FIG. 15 is a cross-sectional view of the illustrative coupling member 1000 taken at line 15-15 of FIG. 14. The coupling member 1000 may be used independently or in combination with any of the coupling members 500, 600, 700, 800, 900 described herein. The coupling member 1000 may include a generally planar body portion 1002 extending from a first end 1004 to a second end 1006 and from a first lateral side 1008 to a second lateral side 1010. The body portion 1002 may have a thickness extending from a front surface 1012 to a back surface 1014. The thickness may vary from the first end 1004 to the second end 1006 and/or from the first lateral side 1008 to the second lateral side 1010. The body portion 1002 may have a generally pentagonal shape. The body portion 1002 may take other shapes, such as, but not limited to, triangular, square, rectangular, polygonal, eccentric shapes, oblong, and the like.

A hook region 1016 may extend from the second end 1006 of the body portion 1002. The hook region 1016 may be configured to receive the handle or other hanging mechanism of the reservoir 302. The hook region 1016 may include a curved region such as a generally “U”, “J”, or horseshoe shaped region 1018. The “U” shaped region 1018 may include a first leg 1020, a curved bridge portion 1022, and a second leg 1024. The second leg 1024 may be laterally spaced from and extend generally parallel to the first leg 1020. The curved bridge portion 1022 may connect the first leg 1020 with the second leg 1024.

One or more arms 1026a, 1026b may extend laterally from the first lateral side 1008 and/or the second lateral side 1010. In some cases, the arms 1026a-b may extend from the plane of the body portion 1002 (e.g., non-parallel to the plane of the body portion 1002). However, this is not required. The one or more arms 1026a-b may each include a clamp 1028a, 1028b at a free end thereof. The clamps 1028a-b may each define a lateral opening 1030a, 1030b configured to receive a tube set therethrough. A central opening 1032a, 1032b may extend through a thickness of the clamps 1028. In some cases, the clamps 1028 may have a generally “C” shape. The clamps 1028 may be configured to receive an endoscope end of the irrigation supply tubing 328 or an endoscope end of the gas and lens wash supply tubing 336, 338 during equipment change over. It is further contemplated that the clamps 1028 may be configured to receive the fluid supply ends of the irrigation supply tubing 328 or the gas and lens wash supply tubing 336, 338 during a reservoir 302, 330 change out.

The coupling member 1000 may further include a securement member 1034 positioned adjacent to the first end 1004 of the body portion 1002. In some examples, the securement member 1034 may extend from the back surface 1014 of the body portion 1002 in a direction opposite the front surface 1012. The securement member 1034 may be generally centered between the first lateral side 1008 and the second lateral side 1010 of the body portion 1002. However, this is not required. In some cases, a first end 1044 of the securement member 1034 may be even with or in-line with the first end 1004 of the body portion 1002. However, this is not required. In some examples, the first end 1044 of the securement member 1034 may be spaced (in either direction) from the first end 1004 of the body portion 1002.

The securement member 1034 may include a first panel 1036 and a second panel 1038 connected to and extending from the back surface 1014 of the body portion 1002. The first panel 1036 and the second panel 1038 may be laterally spaced from one another. In some examples, the first panel 1036 and/or second panel 1038 may extend at a generally orthogonal angle relative to the plane of the body portion 1002. However, this is not required. The first panel 1036 and/or the second panel 1038 may extend at non-orthogonal angles. A third panel 1040 may extend between the first panel 1036 and the second panel 1038. In some examples, the third panel 1040 may extend generally orthogonal to the first and second panels 1036, 1038. However, this is not required. The third panel 1040 may be spaced from the back surface 1014 of the body portion 1002 to define an opening 1042 extending between a first end 1044 and a second end 1046 of the securement member 1034. The securement member 1034 may have a height (e.g. a distance between the first end 1044 and the second end 1046) less than a height (e.g., a distance between the first end 1004 and the second end 1006) of the body portion 1002. The opening 1042 may be sized and/or shaped to receive another coupling member therethrough, such as, but not limited to any of the coupling members 500, 600, 700, 800, 900, or portions thereof, described herein.

The coupling member 1000 may further include a bracket 1048 extending from the back surface 1014 of the body portion 1002 in a direction opposite the front surface 1012. The bracket 1048 may be generally centered between the first lateral side 1008 and the second lateral side 1010 of the body portion 1002. However, this is not required. Further, the bracket 1048 may be positioned between the second end 1046 of the securement member 1034 and the second end 1006 of the body portion 1002. However, the bracket 1048 may be positioned in other locations as desired. In some examples, the bracket 1048 may be axially aligned with the securement member 1034.

The bracket 1048 may be generally “L” shaped having a first leg 1050 connected to and extending from the back surface 1014 of the body portion 1002. The first leg 1050 may extend at a generally orthogonal angle relative to the plane of the body portion 1002. However, this is not required. A second leg 1052 may extend generally orthogonal from the first leg 1050. However, the second leg 1052 may extend at non-orthogonal angles as desired. The second leg 1052 may be spaced from the back surface 1014 of the body portion 1002 to define an opening or gap 1054. In some examples, the length of the first leg 1050 may be selected to achieve a desired distance 1056 between the back surface 1014 of the body portion 1002 and the second leg 1052. For example, in some cases, the distance 1056 may be sufficient to allow another coupling member to be wrapped underneath the first leg 1050 while also allowing the bracket 1048 to be coupled to a rim or ledge of a shelf 402a-e of the cart 400. This is just an example. The distance 1056 may be varied to allow the coupling member 1000 to be hooked onto varying portions of the cart 400, capital equipment, etc.

In some embodiments, the coupling member 1000 may be formed as a single monolithic structure. In other embodiments, the coupling member 1000 may be formed from two or more components that are subsequently coupled together. The coupling member 1000 may be formed from a variety of different materials including metallic materials, such as, but not limited to, aluminum, stainless steel, metal alloys, and the like, polymeric materials, such as, but not limited to, acrylonitrile butadiene styrene (ABS), polyimide, polyamide-imide (PAI), polyamide, polyether ether ketone (PEEK), or the like, or from polymer materials reinforced with glass fiber, ground mica, nanoclay, graphene, talc, kaolin, wollastonite, and the like. It is further contemplated that the coupling member 1000 may be formed from a material which is chemically resistant to allow the user to use bacteriostatic and bactericidal cleaners on and around the coupling member 1000. In some examples, the coupling member 1000 may be formed, at least in part, with materials having bactericidal properties, such as, but not limited to, ionic-silver, copper or other chemical species or compounds known to inhibit microorganism growth.

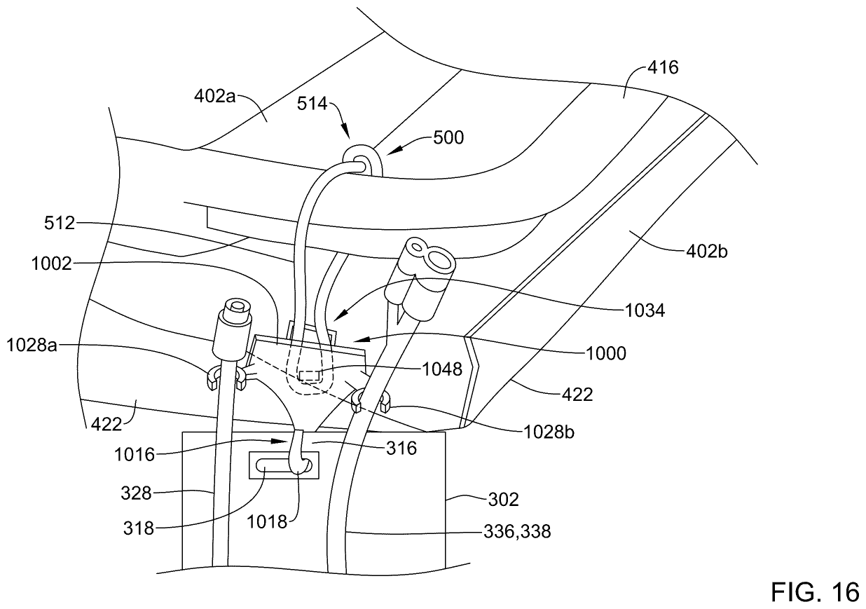

FIG. 16 is a schematic view of the illustrative coupling member 1000 of FIGS. 13-15 secured to a cart 400 and holding a reservoir 302. In the illustrated embodiment, two coupling members 500, 1000 are in use. However, this is not required. Further, it is contemplated that any combination of coupling members may be used, as desired. The first coupling member 500 may be secured to the bar 416 and the second coupling member 1000 secured to the first coupling member 500 and/or the cart 400. To assemble the first and second coupling members 500, 1000, an intermediate portion of the elongate body 502 of the first coupling member 500 may be positioned within the gap 1054 between the bracket 1048 and the back surface 1014 of the body portion 1002 of the second coupling member 1000. The free ends 504, 506 of the elongate body 502 of the first coupling member 500 may be advanced through the opening 1042 of the securement member 1034. For example, the free ends 504, 506 may be advanced through the opening 1042 from the second end 1046 towards the first end 1044 of the securement member 1034.

To couple the first coupling member 500 to the bar 416, the first end 504 of the elongate body 502 may then be positioned on a first side of the bar 416 and the second end 506 positioned on a second side of the bar 416 with the each of the first and second ends 504, 506 above the bar 416. One or both of the first or second ends 504, 506 may be twisted or wound relative to the other to form a twisted region 514. The twisted region 514 may be axially offset from the first end 504 and the second end 506 such that in the twisted configuration the coupling member 500 includes a loop 512, the twisted region 514, a first free end region 516, and a second free end region 518. In some embodiments, the bracket 1048 of the second coupling member 1000 may be secured relative to a shelf 402a-e prior to securing the first coupling member 500 relative to the bar 416. For example, the bracket 1048 may be positioned on upper surface of a raised lip 422 of the shelf 402b such that the second leg 1052 is disposed towards an interior of the shelf 402b and the body portion 1002 is disposed exterior to the raised lip 422. It is contemplated that the intermediate portion of the elongate body 502 that is wrapped under the bracket 1048 may be disposed between the back surface 1014 of the body portion 1002 and an exterior surface of the raised lip 422. However, other configurations or orientations of the first coupling member 500 relative to the raised lip 422 and/or bracket 1048 may also be used.

Once the first and second coupling members 500, 100 are secured relative to the cart 400, the fluid reservoir 302 may be hung from the hook region 1016 of the second coupling member 1000. It is contemplated that the second coupling member 1000 may be used without the first coupling member 500. When not in use, the irrigation tubing 328 may be received within one of the clamps 1028a and the gas and lens wash supply tubing 336, 338 may be received in the other clamp 1028b.

As will be appreciated, the lengths of irrigation, lens wash, gas supply, alternate gas supply tubing may have any suitable size (e.g., diameter). In addition, the sizing (e.g., diameters) of the tubing may vary depending on the application. In one non-limiting embodiment, the irrigation supply tubing may have an inner diameter of approximately 6.5 mm and an outer diameter of 9.7 mm. The lens wash supply tubing may have an inner diameter of approximately 5 mm and an outer diameter of 8 mm. The gas supply tubing may have an inner diameter of approximately 2 mm and an outer diameter of 3.5 mm. The alternative gas supply tubing may have an inner diameter of approximately 5 mm and an outer diameter of 8 mm.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

All apparatuses and methods discussed herein are examples of apparatuses and/or methods implemented in accordance with one or more principles of this disclosure. These examples are not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

In the foregoing description and the following claims, the following will be appreciated. The phrases “at least one”, “one or more”, and “and/or”, as used herein, are open-ended expressions that are both conjunctive and disjunctive in operation. The term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The foregoing discussion has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. One skilled in the art will appreciate that the disclosure may be used with many modifications of structure, arrangement, proportions, materials, components, and otherwise, used in the practice of the disclosure, which are particularly adapted to specific environments and operative requirements without departing from the principles of the present disclosure. For example, elements shown as integrally formed may be constructed of multiple parts or elements shown as multiple parts may be integrally formed, the operation of elements may be reversed or otherwise varied, the size or dimensions of the elements may be varied, and features and components of various embodiments may be selectively combined. The presently disclosed embodiments are therefore to be considered in all respects as illustrative and not restrictive, the scope of the claimed invention being indicated by the appended claims, and not limited to the foregoing description.

The following claims are hereby incorporated into this Detailed Description by this reference, with each claim standing on its own as a separate embodiment of the present disclosure. In the claims, the term “comprises/comprising” does not exclude the presence of other elements or steps. Furthermore, although individually listed, a plurality of means, elements or method steps may be implemented by, e.g., a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second”, etc., do not preclude a plurality. Reference signs in the claims are provided merely as a clarifying example and shall not be construed as limiting the scope of the claims in any way.

What is claimed is:

1. A coupling member for hanging a fluid reservoir relative to an endoscope cart, the coupling member comprising:
an elongate body extending from a first end to a second end;
wherein the elongate body is configured to extend though an opening of a flexible reservoir and around a bar of an endoscope cart.

2. The coupling member of claim 1, wherein the elongate body comprises a flexible wire.

3. The coupling member of claim 2, further comprising a coating disposed over the elongate body.

4. The coupling member of claim 1, wherein free end regions of the elongate body are configured to be twisted together to form a looped region.

5. The coupling member of claim 1, wherein the elongate body further comprises an insertion head adjacent to the second end thereof and plurality of teeth formed in a surface of the elongate body between the first end and the second end and the first end of the elongate body is configured to be inserted into the insertion head to form a looped region.

6. The coupling member of claim 1, wherein the elongate body comprises a flexible material.

7. The coupling member of claim 1, wherein the elongate body is a bistable spring.

8. The coupling member of claim 7, wherein the elongate body is movable from a first elongated configuration to a second curled configuration defining a looped region.

9. The coupling member of claim 1, further comprising a hook coupled to the elongate body.

10. A coupling member for hanging a fluid reservoir relative to an endoscope cart, the coupling member comprising:
an elongate body extending from a first end to a second end;
a first buckle secured adjacent to the first end of the elongate body;
a second buckle secured adjacent to the second end of the elongate body; and
a hook coupled to the elongate body;
wherein the elongate body is configured to wrap around a region of an endoscope cart.

11. The coupling member of claim 10, wherein the elongate body comprises a flexible strap.

12. The coupling member of claim 10, wherein the first buckle is releasably coupled to the second buckle.

13. The coupling member of claim 10, wherein a length of the elongate body is adjustable.

14. A coupling member for hanging a fluid reservoir relative to an endoscope cart, the coupling member comprising:
a generally planar body portion extending from a first end to a second end;
a hook region extending from the second end of the generally planar body portion, the hook region including a curved region;
a securement member extending from a back surface of the generally planar body portion, the securement member including an opening extending therethrough; and
a bracket extending from the back surface of the generally planar body portion;
wherein the hook region is configured to receive a portion of a fluid reservoir.

15. The coupling member of claim 14, further comprising at least one arm extending from a lateral side of the generally planar body portion.

16. The coupling member of claim 15, wherein the at least one arm is configured to receive a tubing.

17. The coupling member of claim 14, wherein the bracket is generally “L” shaped.

18. The coupling member of claim 14, wherein the bracket includes a first leg extending generally orthogonal to a plane of the generally planar body portion and a second leg extending generally parallel to the plane of the generally planar body portion.

19. The coupling member of claim 18, wherein the second leg is spaced a distance from the back surface of the generally planar body portion.

20. The coupling member of claim 14, wherein the coupling member is a single monolithic structure.
